(19) United States
(12) Patent Application Publication
(10) Pub. No.: US 2006/0264751 A1
(43) Pub. Date: Nov. 23, 2006

(54) Protective thin film dressing for therapeutic and diagnostic ultrasound

(76) Inventors: Martin Edmund Wendelken, Elmwood Park, NJ (US); Charles L. Pope, Guliford, CT (US); Howard S. Rosenbaum, Hillsdale, NJ (US)

Correspondence Address: Martin E. Wendelken, 610 Boulevard, Elmwood Park, NJ, 07407, US

(21) Appl. No.: 11/121,800

(22) Filed: May 4, 2005

(51) Int. Cl. A61B 8/00; A61H 1/00

(52) U.S. Cl. 600/439; 601/1

(57) ABSTRACT

A protective thin film adhesive dressing 10 is disclosed for use during diagnostic ultrasound exams and therapeutic ultrasound treatments. Thin film dressing 10 accommodates and covers various shaped transducers 22 and provides a sterile contact surface between a transducer's acoustic window 24 and a body 34, human or animal. Thin film dressing 10 is transparent, ultra light weight and does not change the shape or dimensions of an ultrasound probe 22 during use. Ultrasound wave freely traverse thin film dressing 10 without have a contact media such a gel 20 between acoustic window 24 and the inner surface of thin film dressing 10.

### 1. FIELD OF THE INVENTION

This patent relates to the use of a protective thin film dressing during both diagnostic ultrasound applications and therapeutic ultrasound treatments.

### 2. BRIEF DESCRIPTION OF PRIOR ART AND BACKGROUND OF THE INVENTION

In medicine, diagnostic ultrasound has become one of the most utilized imaging methods used to diagnose and monitor a great number of medical conditions. In addition, ultrasound is also utilized for therapeutic conditions. Both diagnostic and therapeutic applications employ a transducer (ultrasound probe) which generates and sends ultrasound waves into a body. It is well established that air is a poor conductor of ultrasound waves. When utilizing either diagnostic, therapeutic, or a combination of both modalities of ultrasound, a contact media is placed between a transducer and a body. This contact media, usually in the form of a gel or a standoff pad, couples a transducer and a physical surface of a body. Once coupled, the gel serves as an acoustic window which allows unimpeded transfer of ultrasound waves emitted from the transducer into a body. In therapeutic applications, ultrasound waves are primarily sent into a body. In diagnostic ultrasound applications, pulsed ultrasound waves are sent into a body with the intention of generating echoes. Echoes are generated as ultrasound waves encounter various anatomical structures. The generated echoes travel in the opposite direction of the ultrasound waves emitted from the transducer. The echoes then exit the body through the coupling gel. After exiting the body, the same transducer that generated the ultrasound waves, now capture the echoes which are then processed and compiled into an image.

The process of utilizing ultrasound waves in both therapeutic and diagnostic applications requires that physical contact occurs between a transducer and a body. Transducers are expensive, sophisticated, delicate instruments that employ fragile piezoelectric crystals to generate ultrasound waves. In practice, these probes are not sterilized each time they are used on a body. They are not normally used on open wounds or surfaces where infections may occur. After a diagnostic exam or therapeutic treatment, the transducer is typically cleaned with water and a damp cloth or an antiseptic solution in association with a damp cloth. Many manufacturers warn of possible damage to the transducer and instruct to avoid using most cleaning solutions available. Most transducers are not designed to be placed in an autoclave of any type, sterilized using gas, or radiation. Present cleaning methods of ultrasound transducers used to acquiring diagnostic images or deliver therapeutic treatments provide little protection against contaminated probes.

Some prior art does reveal various methods of protecting a person from transfer of micro-organisms during a diagnostic exam. One example is U.S. Pat. No. 6,402,695 (2002) Grimm, who employs a conventional disposable condom which is placed over an ultrasound transducer. Grimm's probe cover includes a tube that allows gel to pass from the distal end of the condom when the probe is inserted into a body. Another example is U.S. Pat. No. 5,795,632 (1998) Buchalter, which discusses a double layer protective probe cover for use during an endocavity or interoperative procedure. Still another example is a condom like probe cover is described in U.S. Pat. No. 5,997,481 (1999) Adams et al. which incorporate a gel reservoir within the distal end of the cover. The gel reservoir is accessed when a probe is inserted and presses against the end of the cover containing the gel. In U.S. Pat. No. 6,051,293 (2000) Weilandt, discloses another protective probe sheath which has a number of chambers incorporated in the sheath. The described chamber contains gel or various liquids which come in contact with an ultrasound probe in order to provide an acoustic window during the application and use of the sheath. Another invention is described in U.S. Pat. No. 6,719,699 (2004) Smith, who applies a double backed adhesive hydrogel membrane to an ultrasound probe which is then inserted into a sleeve or cover. Smith's adhesive membrane is a fixed coupling device that is intended to adhere to a transducer. This same transducer with the adhesive hydrogel membrane attached, is inserted into a sheath (cover) whereby the adhesive membrane sticks to the inside surface of the sheath. The double backed adhesive hydrogel membrane is intended to replace loose coupling gels or fluids within the sheath that cover ultrasound probes. Still another cover for an ultrasound probe is disclosed by Marino in U.S. Pat. No. 6,132,378 (2000) where a cup shaped cover with a flexible membrane is attached to an ultrasound probe. Wendelken et al. in U.S. Pat. No. 6,193,658 (2001) describes a method protecting an area of a body during an ultrasound exam or treatment by covering the area with a flexible film dressing.

Although these methods are fine for their intended purposes there are some inherent shortcoming with each of the described methods or covers. Grimm, Buchalter Weilandt, and Adams, et al, methods all require transmission gel or other contact media within their covers. Smith eliminates the free form gel and adds an adhesive hydrogel to the end of a transducer which is placed within a sheath. Using a sheath requires that an operator places some transmission gel inside the sheath along with the transducer. Few sheaths are custom made to tightly fit a transducer. Most of the sheaths that are used are loose, non-conforming covers that contain gaps which allow air inside. Many times, rubber bands are employed to help hold the sheath over the probe and transmission cable as described by Grimm. Air within the sheath prevents the transmission of ultrasound waves and will at times interfere with image quality or treatment. Also, using a sheath to cover a transducer is cumbersome and slippery which greatly increases the risk of dropping a fragile probe. In addition, sterile sheaths are expensive to use. The application of a sheath over a transducer using Smith's doubled sided adhesive hydrogel membrane increased the probability of the cover sticking to the side making it difficult to apply.

Standoff pads are also used to couple a transducer and a body when using ultrasound. Standoff pads are composed of rubber type material and provide an acoustic window with properties similar to gel. Transducers are made by a number of manufactures each of which has their own design and shape. The cost of designing and manufacturing custom molded standoffs are expensive because standoff pads can only be used on the probe that it was designed for. These standoff pad are designed and intended to be used many times before they fatigue or exhibit artifacts. Previous art fails to discuss decontamination of such devices. Other non-customized standoff pads are available such as those manufactured by Parker Labs, New Jersey, Aquasonic™ Gel Pad. These gel pads are usually disposable. They are not attached to the transducer and are used by placing the disc between the transducer and a body. These standoff pads do provide some protection against contamination provided that they are used once and then discarded. The disposable standoff pads do have some inherent negative issues associated with their use. Disposable standoff pads are cumbersome because an operator is required to hold the standoff pad during an exam or treatment in addition to the transducer. Diagnostic exams and therapeutic treatments are more difficult to perform when using a standoff pad of this type. For example the examiner needs to move both the transducer and standoff pad when examining a large area of the body like an abdomen. Using both hands prevents an examiner from making adjustments to an ultrasound device while holding the probe and standoff pad.

Other prior art such as Marino's discloses disposable covers that fits over the end of therapeutic ultrasound probes. These disposable covers are intended to snap on over the end of a probe. Although these covers provide some protection and are disposable, they must be design for specific transducers. The method of attachment as described in this disclosure discusses a cover that attaches to a ridge which may transducers do not have. Further, a probe cover of this type can only fit on one specific type of transducer shape. This requires an expensive mold to be made for each type of transducer greatly increasing medical costs. When a cover like Marion's is utilized, if the cover is not attached properly or exactly over transducer surface, gapping will occur causing poor image quality or decreased therapeutic value. Wendelken et al. thin film dressing is use to cover a wound or area of skin during an ultrasound exam or treatment. This method greatly reduces the risk of transferring contaminants or germs between a transducer and a body. Although this method is fine for its intended purpose, large areas of skin such as an abdomen would need to be covered to be an effective method of preventing transfer. This at times will prove to be difficult to achieve when applying a large piece of thin film dressing on a body.

Although all the above methods have merit and are useful for their intended purposes, no one method discusses the application of a protective thin film dressing directly over the exterior surface of a transducer.

### 3. OBJECTIVES AND ADVANTAGES

The present invention provides a protective thin film dressing which has a number of advantages over prior art.

A principal objective of this invention is to provide a dressing to seal the external surface of a transducer which comes in contact with a body.

Another objective is to provide a thin film dressing that allows unimpeded transmission of ultrasound waves through the film dressing.

Still another objective is to provide a thin dressing that allows unimpeded transmission of echoes generated by ultrasound waves through the film dressing.

Yet another objective to use a thin film dressing which is transparent (acoustically invisible) on an ultrasound monitor when imaging a body.

Still another objective to utilize a thin film dressing that has the ability to conform to transducers of different shapes and sizes.

Another objective is to take advantage of a thin film dressing's tinsel strength and elastic quality for sealing purposes.

Another objective is to use a sterile thin film dressing which will provide a clean surface to come in contact with a body.

Yet another objective is to use a thin film protective dressing which extremely light in weight.

Still another objective is to use a protective thin film dressing that will not change the shape of transducer when utilized.

Another objective is to provide a low cost disposable thin film dressing that eliminates the risk of transferring micro-organisms between a body and a transducer.

Another objective is to allow diagnostic exams and therapeutic treatment using a transducer sealed with a thin film dressing over open wounds.

Yet another objective is to allow diagnostic exams and therapeutic treatment using a transducer sealed with a thin film dressing over large surface areas of a body instead of covering a body with a protective dressing.

Still another objective is to eliminate the need of a contact media between the surface of a probe and a thin film dressing.

### BRIEF DESCRIPTION OF DRAWINGS

The above and other objects of this invention may be more clearly-seen when viewed in conjunction with the accompanying drawings wherein:

FIG. 1A—is an image of a rectangular framed thin film dressing with protective backing in place and Fig. B is a second picture of a rectangular framed thin film dressing with the protective backing removed. Both images of the film dressing shows a central area that lacks an adhesive.

FIG. 2A illustrates a typical linear array transducer noting an acoustic window on its distal end. FIG. 2B, depicts a linear array transducer with a layer of transmission gel applied to the surface of the transducer's acoustic window in addition to a custom molded standoff pad mounted over the end of the transducer with the transmission gel contained within.

FIG. 3 is a linear array transducer that has been covered by a protective sheath containing transmission gel within which serves as a contact media between the acoustic window of transducer and the inner surface of protective sheath.

FIG. 4 is an image of a disposable standoff pad on a body surface, a hand holding the standoff pad while a second hand holds an ultrasound transducer in a typical position when being used.

FIG. 5a illustrates an ultrasound transducer with a thin film dressing being applied over the acoustic window. In FIG. 5b the thin film dressing continues to be applied to the transducer and in FIG. 5c the thin film dressing is pictured to extend to and cover the majority of the external surface area of the transducer.

FIG. 6 is a side view of a transducer with a thin film dressing affixed to the outer surface.

FIG. 7 shows 6 different shaped transducers covered with a thin film dressing.

FIG. 8a depicts an adhesive backed thin film strip dressing and a transducer and FIG. 8b shows a picture of a transducer having only the contact surface covered by the thin film strip.

FIGS. 9a and 9b shows a thin film dressing that has been cut to assist in the application of the dressing over the outer surface of a transducer.

FIG. 10 is an example of a transducer that has a standoff pad mounted over the distal end with a thin film dressing covering the standoff pad and transducer.

### REFERENCE NUMERALS IN DRAWINGS

10 Thin Film Dressing
12 Film Dressing Support Frame
14 Film Dressing Backing Layer
16 Non-Adhesive Area of Film
18 Adhesive area on thin film dressing
20 Transmission Gel
22 Linear Array Transducer
24 Acoustic Window
26 Custom Molded Standoff Pad
28 Sheath/Cover for Ultrasound Probe
30 Rubber Band
32 Disposable Standoff Pads
34 Body Surface
36 Operator's Hand No. 1
38 Operators Hand No. 2
40 Round - Convex Transducer
42 Internal Transducer (Rectal/Vaginal)
44 Convex Transducer
46 Micro-Convex Transducer
48 Square Transducer
50 Adhesive Thin Film Dressing Strip
52 Thin Film Dressing with Slits
54 Thin Film Strip Handling Tabs

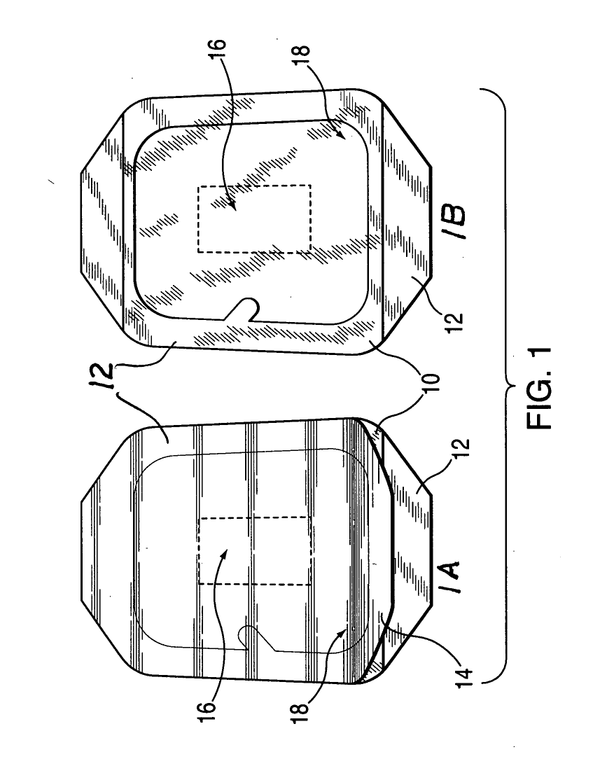

### Overall Description of Embodiment—FIGS. 1 to 10

Directing attention to FIG. 1 of the drawings, two images of a thin film dressing 10 are illustrated. Thin film dressing 10 is typically transparent in nature and normally encased in an outer packaging to keep thin film dressing 10 sterile. FIG. 1 shows two images of thin film dressing 10 both of which have the outer packaging removed. Thin film dressing 10 has two surfaces. In FIG. 1a, thin film dressing 10 shows a support frame 12 on the outer surface and a film backing layer 14 covering the opposite surface. The purpose of film backing layer 14 is to provide protection to an adhesive area 18 applied on the outer edges on one surface of thin film dressing 10. Thin film dressings 10 are extremely thin (ranging in microns) and are very flexible. Support frame 12 provides assistance during application and allows for easy handling. Support fame 12 helps reducing unwanted folding of thin film dressing 10 when thin film dressing 10 is applied to various surfaces.

In FIG. 1b thin film dressing 10 (right) film backing layer 14 has been removed. Note that removal of the film backing 14 exposes an adhesive area 18 primarily found toward the outer edges of thin film dressing 10 leaving a non-adhesive area 16, in this example, in the center of thin film dressing 10. This non-adhesive area 16 is the part of thin film dressing 10 that is in contact with an acoustic window 24 found on ultrasound transducers 22.

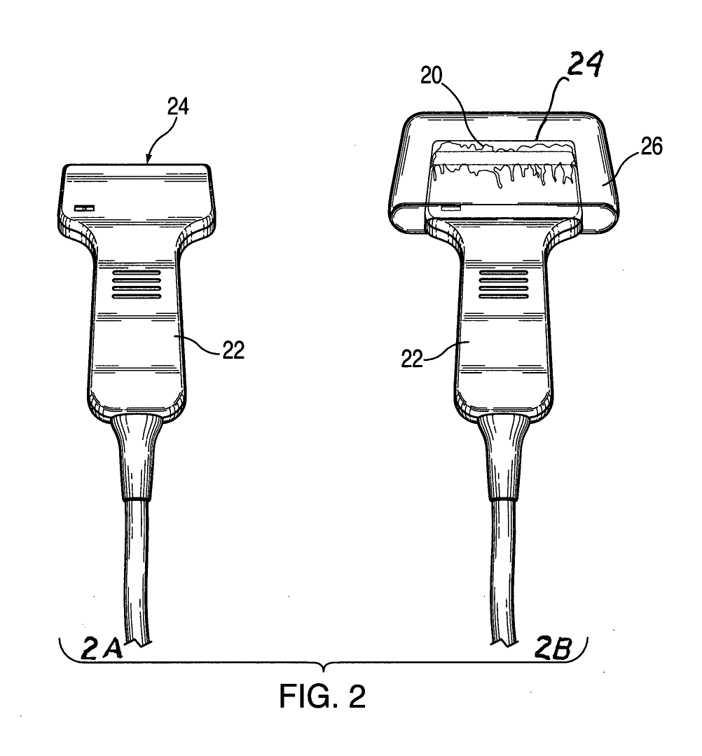

FIGS. 2 A & B—FIG. 2A—is a typical linear array ultrasound transducer 22 that has on one end an acoustic window 24 where ultrasound waves are emitted and echoes are captured. Acoustic window 24 is the contact surface between ultrasound transducer 22 and a body 34. FIG. 2B also illustrates linear array transducer 22 with a custom molded standoff pad 26 mounted over the transducer 22 covering acoustic window 24. A custom molded standoff pad 26 is employed during ultrasound imaging and ultrasound therapy to provide compliance. Additionally, custom molded standoff pad 26 assists in imaging superficial structures during an ultrasound exam. Note that a transmission gel 20 has been applied to the surface of acoustic window 24. Transmission gel 20 is used to insure contact between the inner surface of custom molded standoff pad 26 and acoustic window 24 eliminating air which reduces the flow of ultrasound waves emitted from transducer 22.

Note that custom molded standoff pad 26 increases the size and weight of transducer 22. Further, many custom molded standoff pads 26 are not translucent which interferes with visualizing an area of anatomy being scanned by a sonographer. This lack of visualization leads to increased difficulty of performing ultrasound guided procedures (i.e. injections) when custom molded standoff pad 26 are utilized.

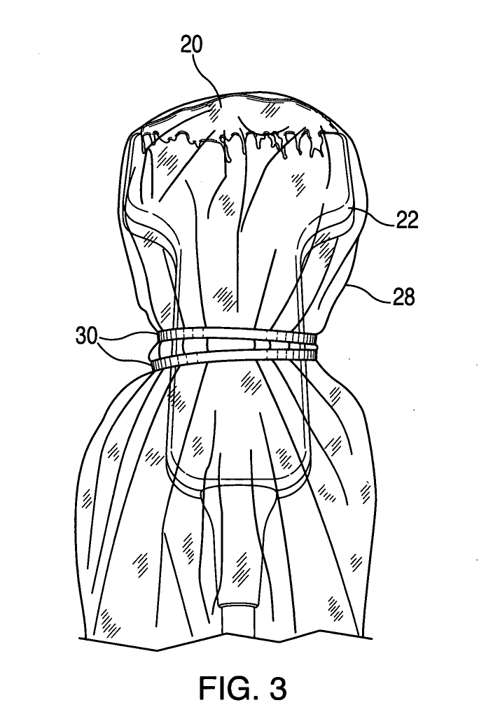

FIG. 3 is a picture of linear array transducer 22 enclosed within a sheath/cover 28. Typically, these sheaths 28 are loose covers that envelope transducer 22 with a primary purpose to keep a person or animal from coming in direct contact with transducer 22. Sheath 28 has inherent problems the least of which is increased difficulty of handling linear array transducer 22. Transmission gel 20 is a necessary component added to the inside of sheath 28 before inserting transducer 22 within. Although transmission gel 20 does help to insure efficient transfer of ultrasound waves from transducer 22 through sheath 28 into a body 34, this same transmission gel 20 causes increased handing problems. Transducer 22 becomes slippery due to transmission gel 20 increasing the probability of dropping transducer 22. Further, few sheaths 28 actually conform to the shape of transducer 22, thus most sheaths 28 require the use of a rubber band 30 to help secure sheath 28 to the outer surface of transducer 22.

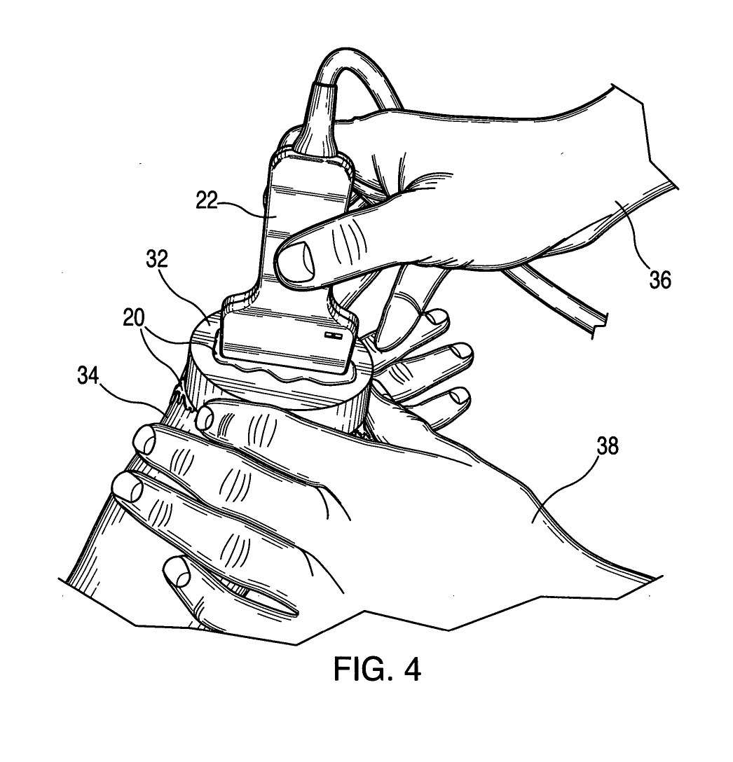

FIG. 4 depicts the hands of a sonographer 36 & 38 holding transducer 22 and a disposable standoff pad 32 while imaging a body 34. Note that left hand 38 holds standoff pad 32 at the same time right hand 36 holds transducer 22. Transmission gel 20 is placed between disposable standoff pad 32 and transducer 22. In addition, transmission gel 20 is also required between disposable standoff pad 32 and body 34. Transmission gel 20 insures free transmission and travel of ultrasound waves from a transducer through disposable standoff pad 32 into body 34. Although disposable standoff pads 32 do provide some protection and avoid direct contact between transducer 22 and body 34 there are some inherent problems using these devices. First of all a sonographer must hold transducer 22 with one hand 36 and disposable standoff pad 32 with another other hand 38. This obviously prevents a sonographer from making important adjustments to an ultrasound machine while imaging or providing treatments. Further, such disposable standoff pads 32 increase the difficulty of imaging large surfaces of a body 34 by having to move both transducer 22 and disposable standoff pad 32.

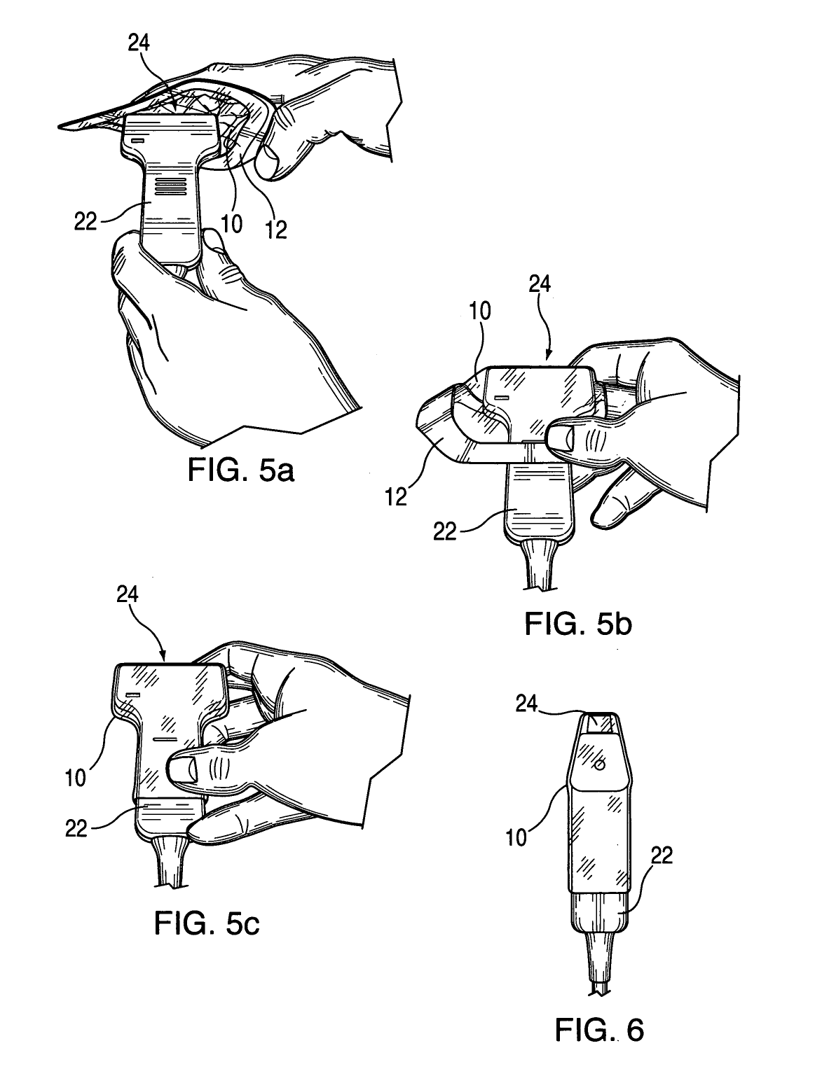

FIGS. 5a, b, c are a series of images showing the application of thin film dressing 10 being applied to the outer surface of linear array transducer 22. The first image FIG. 5a shows thin film dressing 10 is place directly over the distal end of transducer 22 completely covering acoustic window 24. Next FIG. 5b, thin film dressing 10 is draped along the sides of transducer 22 and the film dressing support frame 12 is removed. Transducer 22 is now seen in FIG. 5c with thin film dressing 10 covering almost the entire outer surface of transducer 22. Note that no transmission gel 20 is applied below the surface of thin film dressing 10 during the application to transducer 22.

FIG. 6 shows a side view of transducer 22. Here acoustic window 24 is completely sealed by thin film dressing 10. Again one should note that no transmission gel 20 has been applied to the surface of the acoustic window 24 before application of thin film dressing 10 over acoustic window 24. Thin film dressing 10 is depicted covering almost the entire external transducer 22 and completely conforms to the exterior shape of transducer 22.

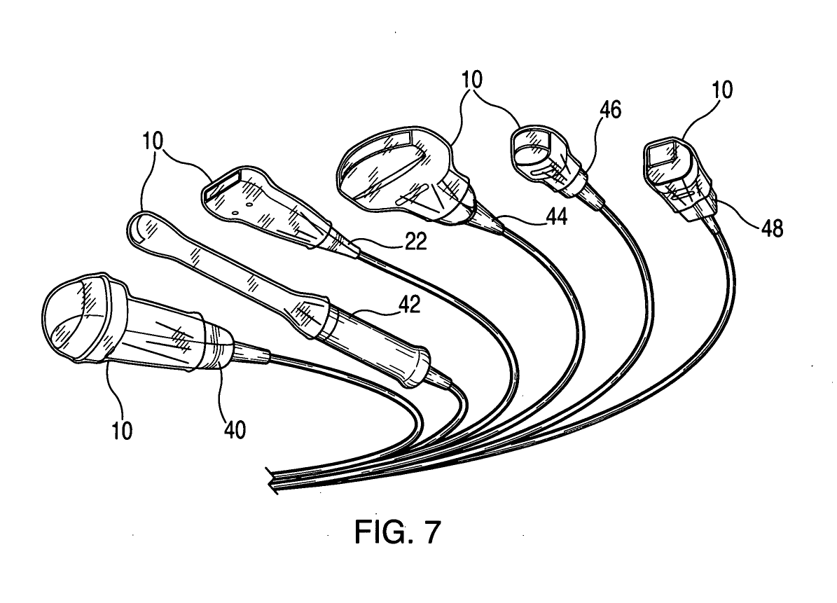

FIG. 7 are examples of a number of different shaped probes with a thin film dressing 10 covering and conforming to the shapes of the various probe types. Starting from the left, a convex transducer 40 having a round body is cover utilizing thin film dressing 10. Next a rectal/vaginal probe 42, followed by a linear array transducer 22 is depicted. Next are two convex probes the first being an abdominal transducer 44 and the other a micro-convex cardiac probe 46. The last probe illustrates a square distal surface 48. All transducers nos. 40, 42, 22, 44, 46, and 48 show ability of thin film dressing 10 to adapt to various contours and shapes.

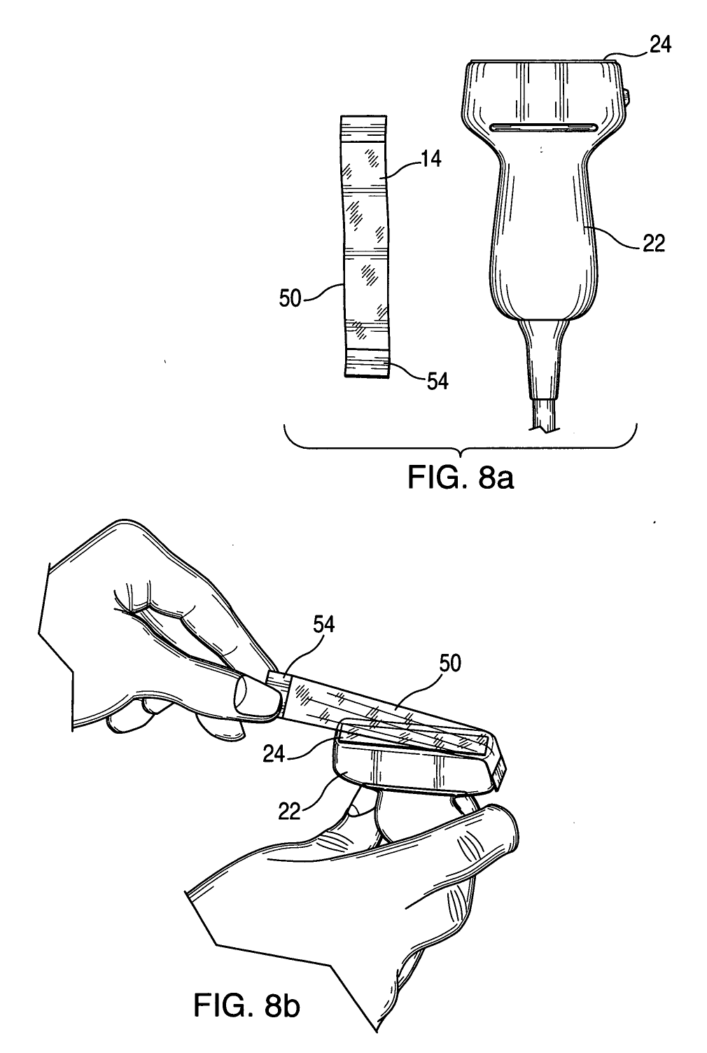

FIG. 8a shows an adhesive thin film dressing strip 50 and linear array transducer 22. Thin film dressing strip 50 has film dressing backing layer 14 along with film dressing support tab 54 on each end. Backing layer 14 covers and protects the adhesive surface of on adhesive film strip 50. Adhesive film strip 50 can be seen being applied to linear array transducer 22 in FIG. 8b. Support Tab 54 located on each end of thin film dressing strip 50 assists in the application (FIG. 8b) of thin film dressing strip 50 over acoustic window 24 located on the distal end of linear array transducer 22. Note the acoustic window 24 which is contact surface of transducer 22 that comes in direct contact with a body 34 being imaged or treated is covered by thin film dressing strip 50. Also note that no contact media such as gel 20 need be applied to between the inner surface of thin film strip 50 and acoustic window 24.

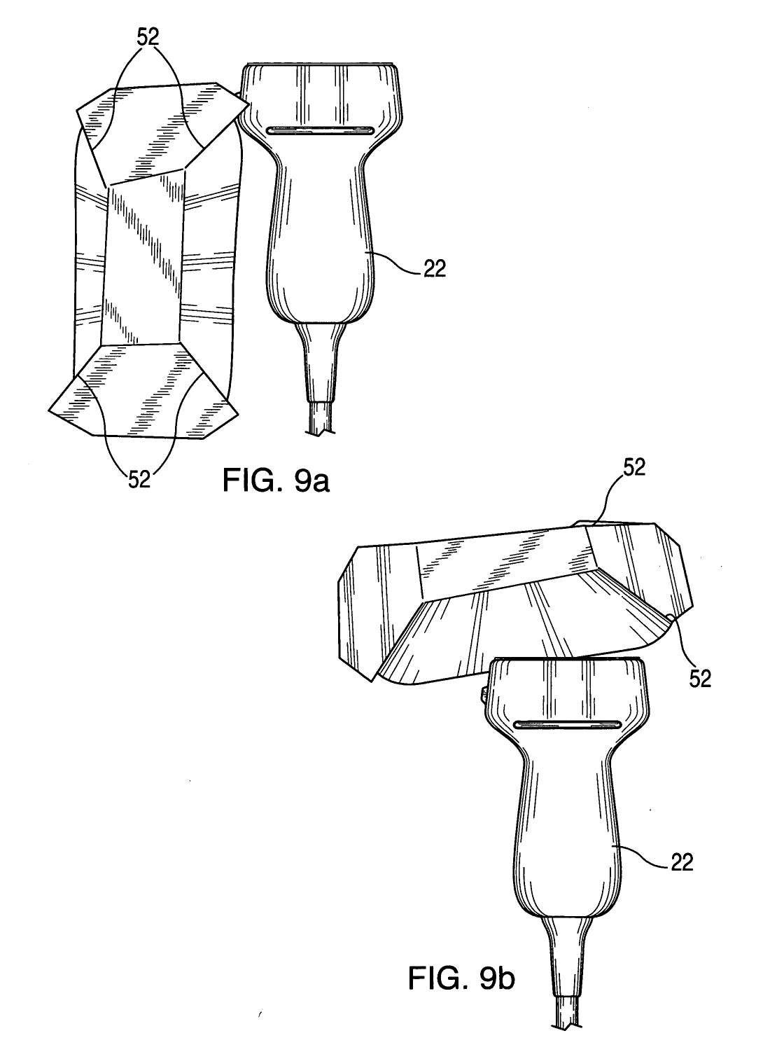

FIG. 9a illustrates a thin film dressing 10 that has a number of slits 52 which will assist in the application of thin film dressing 10 over the external surface of transducer 22. FIG. 9b shows the application of thin film dressing 10 on transducer 22.

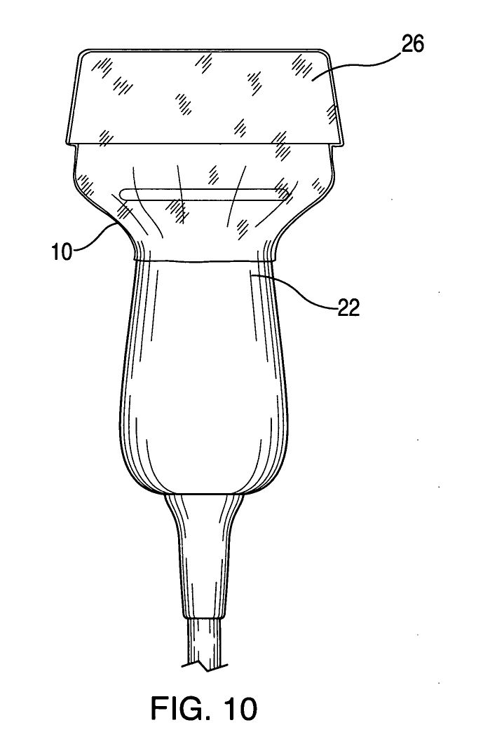

In FIG. 10, thin film dressing 10 has been applied over the external surface of custom molded standoff pad 26 and thin film dressing 10 continues to extend beyond standoff pad 26 onto the external surface of transducer 22.

### Operation Of The Invention—FIGS. 1, 2, 5, 6, 7, 8, 9, 10

FIG. 1—Thin, transparent film dressings 10 are widely used in medical and surgical practices for a number of applications and are offered to the profession in a variety of sizes and shapes. Thin film dressings 10 are generally moisture vapor permeable to permit the covered area to breathe. Thin polymetric films dressings 10 that have a thickness from about 10 to 45 microns are fabricated from polyurethanes such as Goodrich® Estane polyurethane, elastomeric polymetric esters such as DuPont Hytrel® polyester elastomer, and blends of polyurethane and polyester elastomers. Polyurethane and elastomeric polyester films 10 are sufficiently elastic to conform to various contours. These films 10, because of the thinness and flexibility, utilize a delivery system such as a paper frame 12 to assist in the application of the thin film dressing 10. Thin film dressings 10 are sterile and individually packaged. Thin film dressings 10 are coated on one surface with a medical grade pressure-sensitive adhesive 18. The adhesive 18 is protected with a covering of a release paper 14 or other backing 14 which can be readily removed at the time of use. An example of a thin film dressing 10 presently used in an ultrasound application is SoundSeal® protective film dressing marketed by BioVisual Technologies, L.L.C. (NJ, USA) using the Wendelken et al method described in U.S. Pat. No. 6,193,658 (2001) protecting a body 34 during an ultrasound exam or treatment by covering the area with a flexible film dressing 10. Transducers 22 may be used for many years to treat or diagnose a number of conditions. The ability to provide a new, clean contact surface each time transducer 22 is used clearly helps to reduce the risk of transferring micro-organisms between a transducer's 22 acoustic window 24 and a body 34.

After removing thin film dressing 10 from its protective packaging FIGS. 1A & 1B, an operator grasps thin film dressing 10 by one edge holding the thin film dressing 10 by support frame 12. Next, thin film dressing backing 14 is then removed exposing an adhesive coated surface 18 that covers the outer area of thin film dressing 10. In the central portion of thin film dressing 10 exists an area that lacks adhesive 16. This non-adhesive area 16 varies in size and is intended to be large enough to accommodate and cover the entire surface of an acoustic window 24 typically located at the distal end of transducer 22, FIG. 2A. Acoustic window 24 usually is made up of a number of piezoelectric crystals that are covered by a membrane. Its is felt that applying a thin film dressing 10 with an adhesive 18 that affixes to the surface of the acoustic window 24 may cause damage to transducer 22 during the removal of the thin film 10. However, other transducers 22 may be constructed such that acoustic window 24 is designed to have a thin film adhesive dressing 10 with an adhesive 18 in the central portion of thin film dressing 10 applied and removed.

Applying Thin Film Dressing 10 to Transducer 22:

Directing attention to FIG. 5 a-c thin film dressing's 10 non-adhesive area 16 is place over acoustic window 24 (upper left) and held in place with pressure. Next, thin film dressing 10 is stretched, pulled proximal, and applied to each side of transducer 22. Adhesive area 18 on thin film dressing 10 sticks to each side of transducer 22 (upper right). Once thin film dressing is affixed to transducer 10, the operator removes support frame 12 from thin film dressing 10. Lastly, thin film dressing 10 is then applied to the remaining sides of transducer 22. No transmission gel 20 is need below the surface of thin film dressing 10.

A profile view of transducer 22 is depicted in FIG. 6 where one can appreciate thin film dressing 10 which measures in the microns, conforms and covers acoustic window 24 completely. There are no air pockets between thin film dressing 10 therefore no transmission gel 20 is required. Ultrasound wave freely traverse thin film dressing 10. Thin film dressing 10 is transparent therefore an operator may easily observe if any air were to be trapped below thin film dressing surface 10. Further, it should be noted that thin film dressing conforms in shape and adheres to transducer 22 exterior surface in addition to acoustic window 24. FIG. 7 illustrates this ability by conforming and covering various shaped probes 40, 42, 22, 44, 46, and 48. Covering a transducer 22 with ultra light weight thin film dressing 10 in this manner offers protection for patients while at the same time does not change the shape, size, or appearance of transducers 42, 22, 44, 46, 48.

FIGS. 8a and 8b demonstrates the covering of only an acoustic window 24 of transducer 22. A small thin adhesive film strip 50 may be just enough in some cases to cover and protect a patient from the transfer of micro-organisms. Adhesive Strip 50 is constructed with a film backing 14 and support tab 54 on each end to enhance handling. In FIG. 9 a-b various cuts in thin film dressing 10 may provide a distinct advantage during application of thin film dressing 10 to variety of transducers 22, 40, 42, 44, 46, 48 (FIG. 7).

Lastly, FIG. 10 illustrates the use of thin film dressing 10 to cover a custom molded standoff pad 26. Application of thin film dressing 10 is the same as described above. Using a sterile thin film dressing 10 in this fashion provides an additional advantage of having a clean surface available when using a reusable custom molded standoff pad 26.

### SUMMARY AND SCOPE

After reading the fore stated description of the protective thin film dressing 10 for therapeutic and diagnostic ultrasound it becomes apparent that this invention provides a novel method of protecting and covering a transducer 22, acoustic window 24, and standoff pads 26, 32. This same device provides for a cost effective way to reduce the transfer of micro-organisms between a transducer 22 and a body surface 34. This invention offers a number of additional benefits including:
Covering an acoustic window 24 without having to place transmission gel 20 below the film dressing 10 surface.
Covering a transducer 22 with a thin film dressing 10 which will not change the shape of the transducer 22.
Covering a transducer 22 with a thin film dressing 10 that reduces the transfer of micro-organisms between the transducer 22 and the subject being imaged or treated.
Covering the acoustic window 24 with an adhesive thin film 10 or strip of film 50 which does not impede ultrasound waves.
Covering a transducer's acoustic window 24 with a thin film dressing 10 which is invisible and is not seen on an ultrasound display screen.
Covering a transducer 22 with a sterile thin film dressing 10 which will safely allow large areas to be scanned/treated including normal intact skin, cuts, abrasions, and open wounds using ultrasound.
Applying a thin film dressing 10 to cover a standoff pad 26, 32 which provides a clean surface for contacting a body 34 during an ultrasound exam or therapeutic treatment.
Utilizing a thin film dressing 10 or thin film dressing strip 50 to protect both humans and animals during a diagnostic ultrasound exam or therapeutic ultrasound treatment.
   The central concept of a thin film dressing 10 being applied to the external surface of a probe 22 to include covering an acoustic window 24 may be achieved by several varying methods of application without deviation from the intent of this invention. A thin film dressing 10 may be produced in sheets, rolls, or other practical form, with or without a dispenser. A dispenser may be affixed to transducer 10 or a free standing device. Size or exact dimensions of thin film dressing 10 including film 10 thickness may vary providing that thin film dressing 10 remains flexible enough to accommodate different shaped probes 22, 40, 42, 44, 46, 48 and at the same time does not effect the quality of an ultrasound image or treatment. Minor variations in nonessential features of a thin film dressing 10 use on a transducer 22 such as the presence or absence of a protective backing 14, frame 12 or a tab 54 attached to the thin film dressing 10, slits/cuts 52 to modify thin film 10 shape will be inclusive of this application. Further, presence of an adhesive or absence of an adhesive on area of thin film dressing 10 shall all be deemed acceptable variations that still fulfill the intent and use for a protective thin film dressing 10 for ultrasound transducers.

While the invention has been explained by a detailed description of certain specific embodiments, it is understood that various modifications and substitutions can be made in any of them within the scope of the appended claims that are intended also to include equivalents of such embodiments.

Having described our invention what is claimed is:

1. A protective ultrasound transducer dressing for diagnostic and therapeutic ultrasound applications comprising:
a thin film dressing having an adhesive means on one surface thereof thereon;
a transducer having an external surface and an acoustic window positioned thereon; and wherein
said thin film dressing includes an outer surface and an inner surface having an area that lacks said adhesive means whereby said thin film dressing is applied over said transducer's external surface including said transducer's acoustic window, said thin film dressing prevents direct contact between said transducer and a body allowing unimpeded transfer of ultrasound waves.

2. An ultrasonic transducer acoustic window protective dressing for contact with a body for diagnostic applications and therapeutic treatments comprising:
a thin film strip including adhesive means located thereon whereby said thin film strip is applied to a transducer having an acoustic window, said thin film strip covering the external surface of the transducer acoustic window to prevent direct contact between said transducer acoustic window and a body to prevent the risk of infection.

3. The method of applying a thin film dressing to an ultrasound transducer having an acoustic window comprising the steps of:
providing an adhesive thin film dressing having a removable protective backing and outer edges thereon covering an adhesive surface;
providing a support frame along said adhesive film dressing outer edges;
removing and discarding said protective backing to expose said adhesive surface;
applying said adhesive film dressing to the ultrasound transducer covering the acoustic window and adhering to the surface of the transducer, whereby the adhesive film dressing contacts a body during therapeutic and diagnostic procedures; and,
removing the adhesive film from the transducer after use so that a new sterile thin film dressing can be applied for further use.

4. A method of temporarily protecting an ultrasound transducer having an acoustic window from the transfer of micro-organisms or other contaminants during an ultrasound exam or treatment comprising the steps of:
providing a thin film dressing over the transducer to permit unimpeded transfer of ultrasound waves without contact media between the thin film dressing and the acoustic window;
applying the thin film dressing to cover said ultrasound transducer;
said thin film dressing having adhesive on one surface thereof to adhere to the transducer and wherein the thin film dressing seals and protects said ultrasound transducer from contamination during a diagnostic exam, therapeutic treatment or storage.

5. An ultrasonic transducer in accordance with claim 1 wherein:
the thin film dressing includes an outer edge having a support frame mounted thereto to facilitate handling and a removable backing layer in engagement with the inner adhesive layer prior to application to a transducer.

6. An ultrasonic transducer in accordance with claim 5 wherein:
the thin film dressing thickness is in microns.

7. An ultrasonic transducer in accordance with claim 4 wherein:
the transducer is a linear array transducer.

8. An ultrasonic transducer in accordance with claim 1 wherein:
the thin film dressing conforms to the shape of a plurality of different probes.

9. An ultrasonic transducer in accordance with claim 1 wherein:
the thin film dressing comprises a strip having support tabs at each end for ease of handling in the application of said strip over an acoustic window.

10. An ultrasonic transducer in accordance with claim 1 wherein:
the thin film dressing has a plurality of slits, which assist in the application of the thin film dressing over the transducer.
